(19) United States
(12) Patent Application Publication
(10) Pub. No.: US 2008/0004671 A1
(43) Pub. Date: Jan. 3, 2008

(54) VAGUS NERVE STIMULATION VIA ORALLY DELIVERED APPARATUS

(75) Inventors: Rolfe Anderson, Saratoga, CA (US); Pedro E. de la Serna, San Jose, CA (US); Laura Maurer, Mountain View, CA (US)

Correspondence Address: DIEHL SERVILLA LLC, 77 BRANT AVE, SUITE 210, CLARK, NJ, 07066, US

(73) Assignee: ALZA Corporation, Mountain View, CA (US)

(21) Appl. No.: 11/768,400

(22) Filed: Jun. 26, 2007

Related U.S. Application Data
(60) Provisional application No. 60/806,065, filed on Jun. 28, 2006.

(51) Int. Cl. A61N 1/05

(52) U.S. Cl. 607/40

(57) ABSTRACT

Apparatus, systems and methods for stimulation of a vagus nerve of a patient are disclosed. An element that can be deployed and retained inside the patient's stomach without surgery and having a power supply and electrodes is associated with the expandable element. The electrodes are capable of stimulating the vagus nerve while the expandable element remains in the stomach for an extended period of time.

### CROSS-REFERENCE TO RELATED APPLICATIONS

This application claims the benefit of priority under 35 U.S.C. § 119(e) to U.S. Provisional Application Ser. No. 60/806,065, filed Jun. 28, 2006, which is incorporated herein by reference.

### FIELD OF THE INVENTION

Embodiments of the invention generally pertain to methods and apparatus for stimulation of a patient's vagus nerve. More specifically, embodiments of the invention relate to apparatus, systems and methods for stimulation of the vagus nerve via the gastrointestinal tract to control the appetite of a subject.

### BACKGROUND OF THE INVENTION

Various studies and patents have disclosed that electrical stimulation of the vagus nerve can decrease incidence of seizures, help to control hunger, and control the motility of the GI tract. See, for example, U.S. Pat. Nos. 6,611,715; 6,587,719; and 5,188,104; and Hans-Rudolf Berthoud, Multiple Neural Systems Controlling Food Intake and Body Weight, Neuroscience and Biobehavioral Reviews 26 (2002) 393-428; Thomas M. Henry, MD, Therapeutic Mechanisms of Vagus Nerve Stimulation, Neurology 59 (Suppl. 4) September 2002, S3-S14. Severe or morbid obesity is a chronic condition, which may be defined by a body mass index greater than 40 or being at least 100 pounds overweight. Morbid obesity is difficult to treat and affects approximately 3 to 5% of the population. For obese persons, excessive weight is commonly associated with increased risk of cardiovascular disease, diabetes, degenerative arthritis, endocrine and pulmonary abnormalities, gall bladder disease and hypertension. Additionally, such persons are highly likely to experience psychological difficulties because of lifestyle restrictions such as reduced mobility and physical capacity, due to back pain, joint problems, and shortness of breath. Repeated failures of dieting and exercise to resolve the problem of obesity can result in feelings of despair and the development of clinical depression.

Although diets, diet pills, and other weight-reducing plans may be effective for a short period of time, studies have shown that over 96% of those who lose as much as 100 pounds tend to regain that lost weight plus additional pounds within 3 years. Known methods to successfully treat the morbidly obese are invasive and permanent. For example, surgical procedures such as gastric bypass or vertical banded gastroplasty have been used to reduce the size of stomach or decrease absorption in the intestines of severely obese patients. However, these surgeries carry significant risk inherent in all invasive surgeries, and they cannot be modified or reversed.

More recently, systems and methods have been developed for reversibly treating obesity by stimulating the vagus nerve to produce a feeling of satiety, which leads to weight reduction. Vagus nerve stimulation system therapy is already being used in Europe with an implanted device, however, it is not yet approved in the United States by the Food and Drug Administration. Side effects associated with implantable systems include risks associated with surgery including bleeding, infection, and pain. Other problems associated with the implantation of an implanted nerve stimulation device is neurostimulator migration leading to second surgery, pain or seroma at site of neurostimulator, perforation of stomach wall, migration of leads, and allergenic or immune system response to implanted materials. Implantable vagus nerve stimulation systems have also been used to control seizures in patients.

These implantable systems are approximately the size of a wristwatch and have leads that are attached to, or in the vicinity of the vagus nerve. For the systems that are used to control seizures, the device is surgically implanted in the chest area and two leads are routed subcutaneously and attached directly to the vagus nerve. For the systems that are used to control obesity and gastric motility, the device is surgically implanted in the abdominal cavity, and leads are attached to the wall of the stomach. Whether the system is located in the chest area or abdomen, some form of invasive surgery is required to implant or remove the device. Furthermore, a doctor's visit is required to adjust the device for each individuals needs.

Thus, present methods for stimulating the vagus nerve, for both the control of seizures or morbid obesity, are invasive and require some form of surgical device implantation that carries the risks associated with all surgeries in addition to the drawbacks listed above. There is a need to provide methods, apparatus and systems for the stimulation of the vagus nerve that do not involve surgery. Such methods, systems and apparatus may be used to control seizures or treat morbid obesity without the risks, expense and pain attendant with surgical procedures.

### SUMMARY OF THE INVENTION

Embodiments of the present invention provide systems, methods and apparatus for stimulation of the vagus nerve. As such, one embodiment of the invention pertains to an apparatus for stimulating a vagus nerve of a patient comprising an expandable element; electrodes associated with the expandable element, the electrodes configured to generate an electrical field that stimulates the patient's vagus nerve; and a power source in electrical communication with the electrodes, wherein the apparatus is sized and configured to be deployed within the stomach of the patient. In one embodiment, the system or apparatus is deployed for an extended period of time. In one or more embodiments, the expandable element comprises a plurality of elongate members in electromechanical communication with each other. In certain embodiments, the elongate members form a compressible ring structure when the apparatus is deployed inside the patient's stomach. The diameter of the ring structure according to certain embodiments is between about 7 centimeters and 20 centimeters. In certain embodiments, the lengths of the elongate members are sized to fit within an orally ingestible capsule when the ring is compressed. In certain embodiments, the lengths of the elongate members are such that they will fit in size 000 or smaller capsule when the ring is compressed. For example, the length of each elongate member may be less than about 1.8 centimeters.

The apparatus may be packaged for endoscopic delivery to the patient. Alternatively, the apparatus can be configured for oral delivery to the patient. For example, the apparatus can be contained within a capsule that is dissolvable in the patient's gastrointestinal tract.

Another embodiment of the invention pertains to an apparatus for controlling the appetite of a patient comprising an orally administrable element adapted to be retained inside the patient's stomach for an extended period of time and to stimulate the vagus nerve of the patient.

Still another embodiment pertains to an orally administrable apparatus for controlling appetite comprising an expandable ring including a plurality of elongate members connected by electromechanical linkages; a plurality of electrode arrays positioned on the elongate members; and a power source for providing power to the electrode arrays, wherein said electrode arrays generate an electrical field to stimulate the vagus nerve of a patient and suppress the patient's appetite and the apparatus is sized to be delivered orally to the patient. The apparatus may further comprise an oscillator in electrical communication with the elongate members.

Another aspect of the invention pertains to stimulating the vagus nerve of a subject, for example, to control the weight of the subject. In one embodiment, a method of controlling the appetite of a patient is provided which comprises orally delivering means for stimulating the vagus nerve to the stomach of a patient. The means for stimulating the vagus nerve may comprise an expandable ring structure containing electrodes.

In another embodiment, a method of controlling the appetite of a patient is provided, comprising locating an apparatus configured to stimulate the vagus nerve in the stomach of a patient utilizing a non-surgical procedure; retaining the apparatus inside the patient's stomach for an extended period of time; and modulating the apparatus to stimulate the vagus nerve of the patient. The apparatus may comprise an expandable, ring-shaped element.

Advantages and a fuller appreciation of specific adaptations and physical attributes of the present invention can be learned from an examination of the following drawings, detailed description, examples, and appended claims.

### BRIEF DESCRIPTION OF THE DRAWINGS

The present invention is hereinafter described in conjunction with the appended drawings, in which:

FIG. 1 is a schematic showing placement of a vagus nerve stimulation apparatus placed in a patient's stomach;

FIG. 2 is a cross-sectional view taken along line A-A of FIG. 1;

FIG. 3 is a side perspective view of an electrode array portion of a vagus nerve stimulation apparatus;

FIG. 4 is a partial exploded perspective view of FIG. 1;

FIG. 5 is a side perspective view of a vagus nerve stimulation apparatus stored in a capsule for oral delivery;

FIG. 6 is a side perspective view of a vagus nerve stimulation apparatus deployed within an endoscope sheath; and

FIG. 7 is an electrical block diagram for a stimulation system.

### DETAILED DESCRIPTION OF THE INVENTION

Before describing several exemplary embodiments of the invention, it is to be understood that the invention is not limited to the details of construction or process steps set forth in the following description. The invention is capable of other embodiments and of being practiced or being carried out in various ways.

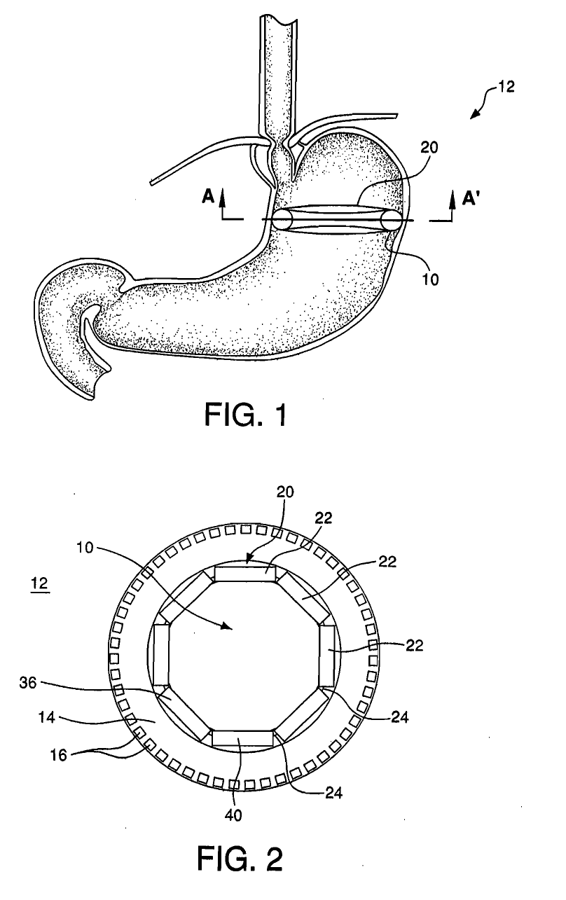

Embodiments of the present invention provide methods, apparatus and systems for vagus nerve stimulation in a patient. An exemplary embodiment of an apparatus for vagus nerve stimulation is shown in FIG. 1, which shows a vagus nerve stimulation apparatus 10 placed in the stomach 12 of a patient comprising an expandable element 20.

Further details of the stimulation apparatus are shown in FIGS. 2-4. The expandable element 20 comprises a plurality of elongate members 22 joined together by electromechanical linkage 24. The vagus nerve stimulation apparatus is retained in the upper gastric cavity by exerting an outward force against the gastric wall 14 of the stomach by the expandable element 20 and adjacent to the vagus nerve loci 16. The outward force can be provided by the electromechanical linkage 24 joining each elongate member to an adjacent elongate member. The force can be provided by making the electromechanical linkage from a suitable shape-memory materials, for example, shape memory polymers, shape memory alloy wire such as NiTi, spring metals, or a hydrogel expanding in a membrane. In the embodiment shown in FIG. 2, the stimulation apparatus 10 comprises an electrode body 28 comprised of at least one of the elongate members, which contains an array of electrodes 30, 32 on the body 28. One of the elongate members has a suitable power source 36 associated therewith and an electronic source control and oscillator 40 is associated with another of the elongate members. Further details on the electronic source control and oscillator 40 are provided below.

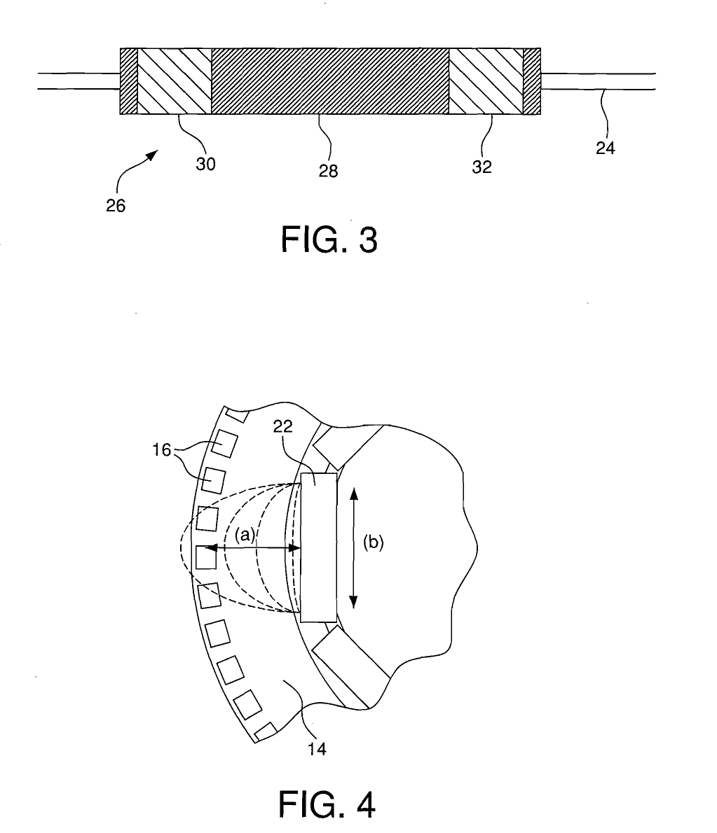

Referring now to FIG. 3, a detailed view of an electrode 26 is shown, which comprises an electrode body 28 carrying a first electrode portion 30 and a second electrode portion 32, thus providing an electrode array. Electromechanical linkage 24 is shown extending from the ends of the electrode body 28.

FIG. 4 shows an exploded perspective view of a portion of the stomach gastric wall 14 and vagus nerve loci 16. The expandable elements 20 are located adjacent the gastric wall 14 and vagus loci 16. A suitable electric field 90 for stimulating the vagus nerve of a particular patient can be determined by a person of ordinary skill in the art. In experiments where the vagus nerve of rats was stimulated, the electric field was approximately 1 V/cm. G. KRÓLCZYK, D. ZUROWSKI, J. SOBOCKI, M. P. S£OWIACZEK, J. LASKIEWICZ, A. MATYJA, K. ZARASKA, W. ZARASKA, P. J. THOR, Effects of Continuous Microchip (MC) Vagal Neuromodulation on Gastrointestinal Function in Rats, JOURNAL OF PHYSIOLOGY AND PHARMACOLOGY 2001, 52, 4, 705-715; J. LASKIEWICZ, G. KRÓLCZYK, D. ZUROWSKI, J. SOBOCKI, A. MATYJA, P. J. THOR, Effects of Vagal Neuromodulation and Vagotomy on Control of Food Intake and Body Weight in Rats, JOURNAL OF PHYSIOLOGY AND PHARMACOLOGY 2003, 54, 4, 603-610. In the configuration shown in FIG. 4, assuming that the electrode-to-electrode spacing (b) and electrode-array-to-stimulation-site spacing (a), in other words, the distance to the vagus nerve loci from the electrode array, are each 1 cm, and homogeneous material properties, a field of approximately 1 V/cm could be achieved at the stimulation site with an electrode array voltage of 2V. This takes into consideration the thickness of stomach mucosa is 1.0 to 1.6 mm, C. H. Huh, et al., Physiol. Meas. 24 (November 2003) N15-N22.

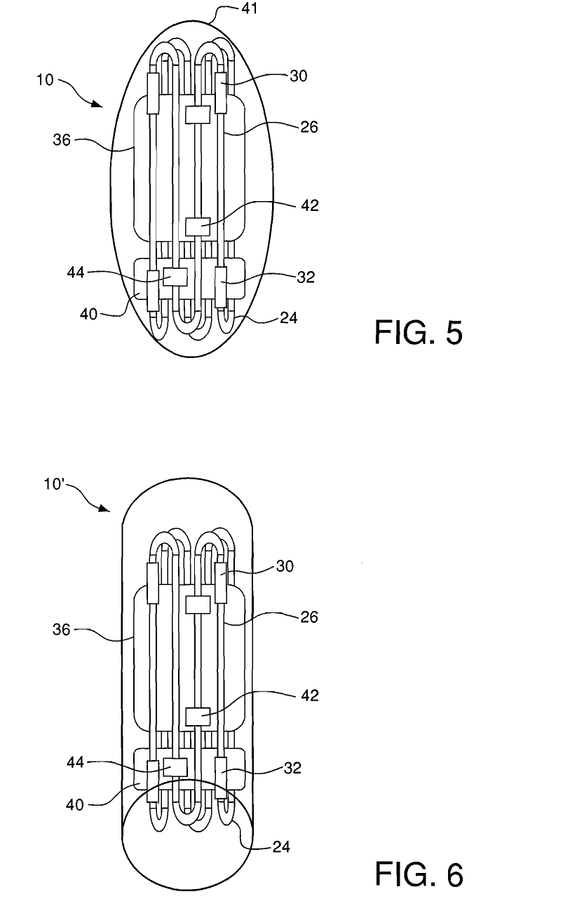

FIG. 5 shows a vagus nerve stimulation apparatus 10 of FIG. 1 folded in capsule 41 for oral delivery to a subject. The apparatus includes the same components as the device in FIG. 1. In FIG. 5, components shown include the power source 36, power source linkages 42, electronic controller and oscillator 40, electronic controller and oscillator linkages 44, electrode arrays 26, each incorporating a pair of electrodes 30, 32, and electromechanical linkages 24. The capsule is a capsule that is adapted to dissolve in the stomach allowing the apparatus 10 to unfold once the capsule 41 is ingested.

Referring now to FIG. 6, another embodiment of a vagus stimulation apparatus 110 is shown. The vagus stimulation apparatus contains essentially the same components as the vagus stimulation apparatus shown in FIG. 1. In the embodiment shown in FIG. 6, the apparatus 110 is folded in an endoscope sheath 105 for endoscopic delivery to a patient. Similar to the system shown in FIGS. 1 and 5, components of the vagus stimulation apparatus 110 include a power source 136, power source linkages 142, electronic controller and oscillator 140, electronic controller and oscillator-linkages 414, electrode arrays 126, each incorporating a pair of electrodes 130, 132, and electromechanical linkages 124. The endoscope sheath 105 is inserted into a patient and retracted to release the apparatus 110 in the stomach allowing it to unfold. The apparatus can be deployed using esophagogastroduodenoscopy (EGD) or upper endoscopy which enables access to the upper gastrointestinal tract of a patient. Such procedures are considered minimally invasive since they do not require an incision into one of the major body cavities and do not require any significant recovery after the procedure.

The power and electrical parameters of the apparatus will now be described. The power needs will be dominated by dissipation through the system resistance is given by Vrms²/R, where Vrms is the root-mean square excitation voltage provided by the system between Electrode A and Electrode B, and R is the lumped resistance between these electrodes.

The system resistance R is given by

R=ρ*b/Area,

where τ is resistivity of the medium, Area is the cross sectional area of conduction, and b is the electrode spacing. Based on tissue measurements, it is estimated that the resistivity of the gastric wall as 1 kΩ cm. See M. R. Prausnitz, Advanced Drug Delivery Reviews 18 (1996) 395-425, wherein based on values are given as the deeper tissue resistance on page 397, the area-based resistivity of deep skin tissues is 100-200 Ωcm². The resistivity is given by 100-200 Ωcm²/0.1 cm=1-2 kΩ cm.

Also, it is estimated that the resistivity of the gastric fluid is 1 kΩcm. Gastric fluids are essentially HCl with a pH of 1 to 3. (J. DeSesso and C. Jacobson, Food and Chemical Toxicology 39 (2001) 209-228). The molar conductivity of gastric fluid is 391, 412, and 421Ω⁻¹ cm² mol⁻¹ (CRC Handbook of Chemistry and Physics, 82^{nd} Edition, D. R. Lide, ed. (2001) Page 5-92), giving an average resistivity of 880 Ωcm.

Assuming an electrode spacing of 1 cm, and an Area of 1 cm², the resulting system resistance R=1 kΩ. This estimate compares favorably with data in U.S. Pat. No. 6,684,104, which is incorporated herein by reference, where 300-800 ohms was reportedly measured between patches attached to the gastric wall (where the electrode spacing is 0.5-2 cm and each patch diameter is 1 to 3 cm).

Therefore, assuming Vrms=1.2 V and R=1 kΩ, the ohmic power dissipation is 1.4 mW. This gives a power dissipation of 1.4 mW per electrode array. A typical apparatus would include up to about five arrays, and multiplying by five arrays per apparatus yields a 7 mW system power dissipation when energized. It will be understood, however, that the invention is not limited to a particular number of arrays. The average power requirements will be lower because of the stimulation duty cycle, and the possibility of deactivating the system during meals and at night. Optimal duty cycle stimulation is reported to be 1:1.8, or on 36% of the time, as disclosed in U.S. Pat. No. 6,587,719. This translates to an average system power (during periods of activation) of 2.5 mW.

The power source is selected so as to be an appropriate size for packaging in an orally ingestible capsule. Alternatively, the power source could be provided by an externally applied electromagnetic field, such as by using inductive coupling.

Existing battery technology has been optimized for other applications to deliver the similar system power. Hearing aid batteries have evolved from mercury cells to zinc-air cells with a very high capacity. While the zinc air batteries offer the best size-performance fit for this application, it is not clear whether there is sufficient air in the gastric cavity for operating a zinc air battery. Alternatives include silver oxide cells, custom batteries, and supercapacitors. Table 1 lists examples of types of batteries, dimensions, capacities and life cycle of the battery at 2.5 mW, based on the calculations above reflecting a 1:1.8 duty cycle.

TABLE 1
Miniature Batteries

| Battery Type | Example | Dimensions | Capacity | Time at 2.5 mW¹ |
|---|---|---|---|---|
| Zinc Air | VARTA | 3.55 mm height × | 60 mAh (1.4 V) | 33.6 hrs |
| | 520020 or | 5.75 mm | | |
| | Duracell DA 230 | diameter | | |
| Silver Oxide | 315 | 1.6 mm height × | 21 mAh (1.55 V) | 13 hrs |
| | | 7.9 mm | | |
| | | diameter | | |
| Mercury | 625 | 5.4 mm height × | 250 mAh (1.35 V) | 135 hrs |
| | | 13.9 mm | | |
| | | diameter | | |
| Super | Maxwell PC-5 | | 5 Farad at 2.5 V² | 3.5 hrs |
| Capacitor | | | | |

Alternatively, an open battery cell can be utilized in which gastric fluids act as electrolytes. In another alternative, the power source can be an external power source based on a capacitive system or inductive coupling. For example, the power source could be strapped to the outside of the patient's abdomen to supply power to the apparatus.

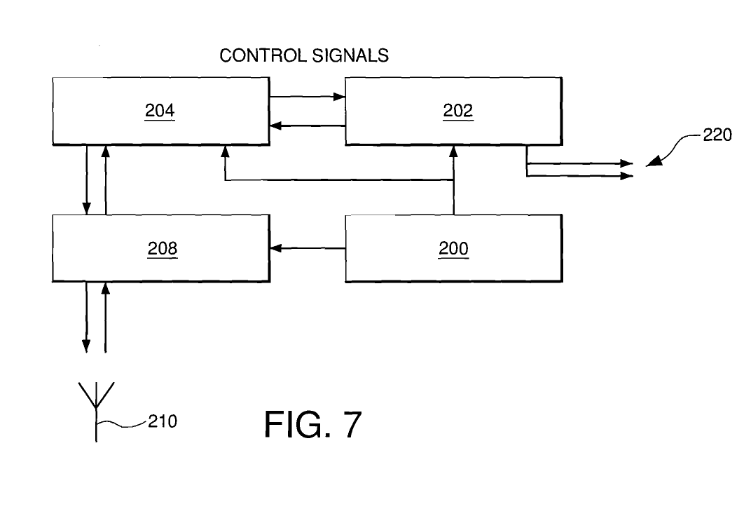

Referring now to FIG. 7, an electrical schematic according to one embodiment of an apparatus is shown. Specific electrical designs of each block are consistent with methods known by those skilled in the art, with the following specifications. Generally, the power supply 200 provides low power operation of the apparatus which includes electrodes 220. According to one embodiment, the oscillator 202 is adapted to generate excitation at a frequency for appetite suppression (e.g. 0.1 to 20 Hz). It will be understood that for other applications that involve stimulation of the vagus nerve, a different frequency may be desired. The oscillator creates a periodic wave in accordance with one aspect of the present invention. The periodic wave can be a square wave, a saw tooth wave, pulsed wave, decaying pulse, a sine wave, or any other type of wave. The wave may also have a DC offset or the DC offset may be set at zero.

The controller 204 can provide control signals to control the duty cycle of the wave generated by the oscillator 202, programmed stimulation, and intensity control of the wave generated by the oscillator 202. The controller 204 and the oscillator 202 can be integrated into one component as shown in the Figures. Alternatively, these components can be discrete elements. Communications 208 may be provided to provide induced or generated power. Antenna 210 may be provided to send and receive signals from communications to the apparatus. The communications 208 may be integrated into the stimulation apparatus, or alternatively, they may be remote from the apparatus to provide for control of the device outside of the body of the patient.

The excitation frequency can be any suitable frequency, and an exemplary range of frequencies is between about 0.1 to 20 Hz. The duty cycle of the apparatus according to one embodiment is between about 1:1 and 100:1. Programmed stimulation can be provided according to one or more embodiments of the invention wherein the apparatus can be programmed to simulate during meal time on a daily basis or based on a custom program according to the patient's needs. The communications can continually monitor the intensity, cycle time, and programming of the apparatus.

The oscillator, controller, and communications can be readily integrated into a microchip as is known in the art. For example, radio frequency identification (RFID) tags include an RF transceiver and coded reporting functions in a package the size of a grain of rice. Also, FPGA (field-programmable gate array) and ASIC (application specific IC) technologies provide for over about 5,000 circuit elements per square cm. For example, Xilinx 4VFX12 FPGA (field programmable gate array) incorporates over 12,000 logic cells in an area 17×17 mm.

In use, the apparatus of the present invention can be administered to a patient by administering a capsule containing the apparatus or by endoscopic delivery as discussed above. When the apparatus is in place inside the patient's stomach, the electrical components are placed in communication and are adapted to generate an electrical output signal in the form of a sequence of pulses, with either predetermined parameter values or values programmable by the attending physician within predetermined, programmed ranges for treating the disorder. The electrodes, which are adjacent the vagus nerve of the patient when the apparatus is deployed in the stomach, apply the programmed output signal to the patient's vagus nerve. Calibration of the overall treatment system for a particular patient can be performed by telemetry by means of an external programmer to and from the implant. The apparatus may be externally programmed for activation upon occurrence of a predetermined detectable event, or, instead might be periodically or continuously activated, to generate the desired output signal with parameter values programmed to treat obesity by modulating vagal activity so as to produce a sensation of satiety. If adjustment to the stimulation is required, the frequency of the stimulation can be adjusted.

Suitable methods, devices and systems for controlling system for stimulation of the vagus nerve are disclosed in U.S. Pat. Nos. 5,263,480 and 6,587,719, the entire contents of which are incorporated herein by reference.

After the system has been utilized for an extended period of time, for example, about a few days or as long as about one to two weeks or longer, the apparatus can be removed using an endoscope. Alternatively, the apparatus can be designed to be excreted through GI tract after use by manufacturing portions of the apparatus using bioerodible elements. Bioerodible elements can be provided which dissolve inside the patient's stomach after a predetermined period of time. Examples of bioerodible elements, include, but are not limited to, polyglycolide (PGA), polylactide (PLA), poly-caprolactone, poly-dioxanone, and poly-lactide-co-glycolide, either alone or combined as co-polymers. Of these, PGA has the strongest flexural modulus (7.0 Gpa). Additional examples of which are disclosed in aforementioned U.S. Pat. Nos. 4,758,436 and 5,047,464. Once the device has been dosed, it will deploy and retain in the GI tract, preferably the stomach. During this retention period the system will be in contact with the lining of the GI tract. During this contact period, electrical energy can be pulsed through the wall thus stimulating the vagal nerve.

Suitable materials to manufacture the apparatus according to the present invention include polymers such as polyethylene, polypropylene and similar materials. Bioerodible materials such as the materials listed above may also be utilized. As noted above, the electromechanical linkages can be made from a suitable conductive shape memory metal such as nickel titanium. The expandable element could be in a variety of forms such as the polygonal expandable element comprised of elongate members linked by electromechanical linkages. Alternatively, the expandable element could be in the form of an inflatable tube or ring. Additional examples of expandable elements that are retained inside the patient's stomach over a controlled, predictable and extended period of time are described in U.S. Pat. Nos. 5,062,829; 4,758,436; 5,047,464, the entire contents of which are incorporated by reference. As used herein, the term “expandable” refers to an element or a member that can increase in dimensions in at least one direction. Expandable elements could further be provided by balloon type structures, and polymer sheets.

In addition to the expandable elements described immediately above, the apparatus of the present invention could be retained in the stomach using a suitable gastric retention system used in drug delivery. Thus, the gastric retention system could include floating systems based on one or more hydrodynamically balanced system incorporating buoyant materials enabling the device to float; effervescent systems that use gas-generating material such as carbonates incorporated into the element which react with gastric acid and produce carbon dioxide, which allows them to float; raft systems incorporating alginate gels which have a carbonate component that reacts with gastric acid to form bubbles in the gel, enabling floating.

In one embodiment, the diameter of the device when deployed and expanded in the stomach is from about 7 to 20 cm. According to one or more embodiments, the elongate members are about 0.5 to 5 cm in length and preferably approximately 1.8 cm in length (0.7 inches to fit into size 000 or smaller capsule, for example size 0 capsule L=0.85″). Other suitably sized capsules such as size 00 capsules may be utilized, which have been used for imaging of the gastrointestinal tract.

Thus, according to embodiments of the present invention, apparatus, methods and systems for controlling appetite in a reversible and noninvasive or minimally invasive manner by stimulating the vagus nerve from the inside of the stomach are provided. The apparatus can be an orally delivered electronic device that provides an electrical field to the surrounding tissue and thereby stimulates the vagus nerve to suppress appetite. The structure of the apparatus can be in the form of a compressible and expandable ring structure comprised of elongate members connected by electrical or electromechanical linkages. Once the device is deployed in the stomach, it will be retained in place, similar to a gastric retained drug delivery device. The expanded ring structure will contact the stomach walls to provide for retention of the device.

It will be apparent to those skilled in the art that various modifications and variations can be made to the present invention without departing from the spirit or scope of the invention. Thus, it is intended that the present invention cover modifications and variations of this invention provided they come within the scope of the appended claims and their equivalents.

1. An apparatus for stimulating a vagus nerve of a patient comprising:
an expandable element;
electrodes associated with the expandable element, the electrodes configured to generate an electrical field that stimulates the patient's vagus nerve; and
a power source in communication with the electrodes, wherein the apparatus is sized and configured to be deployed within the stomach of the patient.

2. The apparatus of claim 1 wherein the expandable element comprises a plurality of elongate members in electromechanical communication with each other.

3. The apparatus of claim 2 wherein the electrodes are associated with a portion of the elongate members.

4. The apparatus of claim 3 wherein the elongate members form a ring structure that unfolds when the apparatus is deployed inside the patient's stomach to provide an expanded apparatus, wherein the diameter of the expanded apparatus is between about 7 centimeters and 20 centimeters.

5. (canceled)

6. The apparatus of claim 4 wherein the length of the apparatus is sized to fit within an orally ingestible capsule when the ring is compressed.

7. The apparatus of claim 4 wherein the length of each elongate member is less than about 1.8 centimeters and is sized to fit in a size 000 or smaller capsule when the ring is compressed.

8. (canceled)

9. The apparatus of claim 1 wherein the apparatus is packaged for endoscopic delivery to the patient.

10. The apparatus of claim 1 wherein the apparatus is configured for oral delivery to the patient.

11. The apparatus of claim 1 wherein at least a portion of the apparatus is manufactured from a bioerodible material.

12. The apparatus of claim 1 wherein the power source is integrated within the expandable element.

13. The apparatus of claim 1 wherein the power source is external to the expandable element.

14. An apparatus for controlling the appetite of a patient comprising an orally administrable system adapted to be retained inside the patient's stomach and to stimulate the vagus nerve of the patient.

15. The apparatus of claim 14 further comprising an expandable element configured to increase in dimension in at least one direction upon deployment into the patient's gastrointestinal tract and to remain inside the patient's stomach.

16. The apparatus of claim 15, further comprising means for generating a frequency to stimulate the vagus nerve of the patient.

17. The apparatus of claim 15, wherein the system includes a gastric retention means for retaining the system inside the patient's stomach.

18. An orally administrable apparatus for controlling appetite comprising:
an expandable ring including a plurality of elongate members connected by electromechanical linkages;
a plurality of electrode arrays positioned on the elongate members; and
a power source for providing power to the electrode arrays, wherein said electrode arrays generate an electrical field to stimulate the vagus nerve of a patient and suppress the patient's appetite and the apparatus is sized to be delivered orally to the patient.

19. The apparatus of claim 18 further comprising an oscillator in electrical communication with the elongate members.

20. The apparatus of claim 18, wherein the electromechanical linkages are manufactured from a shape memory material.

21. A method of controlling the appetite of a patient comprising orally delivering means for stimulating the vagus nerve to the stomach of patient.

22. The method of claim 21 wherein the means for stimulating the vagus nerve comprises an expandable ring structure.

23. A method of controlling the appetite of a patient comprising:
locating an apparatus configured to stimulate the vagus nerve inside the stomach of the patient;
retaining the apparatus inside the patient's stomach for an extended period of time; and
modulating the apparatus to stimulate the vagus nerve of the patient.

24. The method of claim 23 wherein the apparatus comprises an expandable ring-shaped element.

25. (canceled)

26. The method of claim 24, wherein the ring-shaped expandable element is inflatable.

27. The method of claim 24, wherein the ring-shaped expandable element comprises a ring including a plurality of elongate members connected by electromechanical linkages.

28. The method of claim 23, wherein the locating step is performed with an endoscope.
